(19) United States
(12) Patent Application Publication
(10) Pub. No.: US 2017/0191121 A1
(43) Pub. Date: Jul. 6, 2017

(54) Biochip hybridization device

(71) Applicant: Ningbo University, Ningbo (CN)

(72) Inventors: Xiurong Su, Ningbo (CN); Jun Zhou, Ningbo (CN); Dijun Zhang, Ningbo (CN); Yanyan Li, Ningbo (CN)

(21) Appl. No.: 15/466,573

(22) Filed: Mar. 22, 2017

(30) Foreign Application Priority Data
Apr. 28, 2016 (CN) 201610274213.2
Apr. 28, 2016 (CN) 201620370347.X

(51) Int. Cl. C12Q 1/68; B01L 3/00; B01L 7/00

(52) U.S. Cl. CPC C12Q 1/6834; B01L 7/00; B01L 3/508; B01L 2300/0819; B01L 2300/12; B01L 2200/025

(57) ABSTRACT

A biochip hybridization device includes a first hybridization box and second hybridization boxes. Multiple placement cavities for respectively placing the second hybridization boxes are horizontally provided in the first hybridization box. Each second hybridization box includes a substrate which is horizontally arranged, wherein: multiple observation windows, which are concave downward, are arranged on the substrate side by side; a sample addition hole which penetrates through the substrate is provided at an edge of each observation window; supporting arms are respectively arranged at two sides of a bottom surface of the substrate and vertical to the substrate; a sliding slot is provided on an inner wall of each supporting arm and is concave towards a direction of an outer wall of the corresponding supporting arm; and a width of the sliding slot matches a thickness of a biochip. The second hybridization box has a simple structure.

### CROSS REFERENCE OF RELATED APPLICATION

The application claims priority under 35 U.S.C. 119(a-d) to CN 2016203703 47.X, and CN 201610274213.2, filed Apr. 28, 2016.

### BACKGROUND OF THE PRESENT INVENTION

Field of Invention

The present invention relates to a technical field of biochip, and more particularly to a biochip hybridization device.

Description of Related Arts

With the rapid development of the biotechnology and the related disciplines, the biochip is more and more widely applied. The biochip is applied to hybridize with the marked biological sample, and then the number of the target molecules in the sample is determined through detecting the strength of the hybridization signal.

Chinese patent application, CN200610114412.3, disclosed a biochip hybridization box, comprising a base, a cover, a clamping element, and a support. When using the hybridization box, a biochip is firstly arranged on the support, and then a substrate is arranged on the biochip, wherein the biochip is separated from the substrate. When adding a sample into a reaction area through a micropipette, because of a relatively small diameter of the injection hole, a tip of the conventional pipette easily drives the substrate to move, causing the substrate being away from the surface of the biochip, namely, it is easy to generate the sliding of the biochip, thereby leading to a failing hybridization. In order to avoid sliding of the biochip, some operators inject the reagent with the micropipette having the smallest tip for many times, causing the bubbles entering the reaction area, thereby increasing a failure probability of the hybridization. Moreover, after finishing arranging the biochip and the substrate, it is required to arrange the biochip and the substrate in a constant-temperature environment, such as the constant-temperature water, the thermotank and the hybridization oven, meanwhile the cover is arranged on the substrate, the cover and the base are sealed and fixed by the clamping element, and the whole biochip hybridization box is arranged in the constant-temperature water or other corresponding devices. However, the above-described sealing and fixing manner, and the structure thereof are complex; when sealing and fixing, the clamping element generates a certain extrusion force, and the sample and the reagent in the reaction area of the biochip may be extruded to outside of the reaction area because of an effect of the extrusion force, thereby affecting the reaction of the biochip; meanwhile, the above hybridization box has a large occupied area and a relatively high cost; and moreover, the constant-temperature condition is generally realized through an additional device, causing a relatively high experimental cost.

### SUMMARY OF THE PRESENT INVENTION

An object of the present invention is to provide a biochip hybridization device which has a simple structure, a convenient operation and a low cost, and is portable. The biochip hybridization device provided by the present invention is able to effectively avoid sliding of a biochip, implement hybridization of multiple biochips at the same time, and provide a constant-temperature and constant-humidity hybridization environment, and has a high hybridization success rate.

Technical solutions of the present invention are described as follows.

A biochip hybridization device comprises a first hybridization box and second hybridization boxes, wherein: multiple placement cavities for respectively placing the second hybridization boxes are horizontally provided in the first hybridization box; each second hybridization box comprises a substrate which is horizontally arranged; multiple observation windows, which are concave downward, are arranged on the substrate side by side; a sample addition hole, which penetrates through the substrate, is provided at an edge of each observation window; two supporting arms are respectively arranged at two sides of a bottom surface of the substrate and vertical to the substrate; a sliding slot is provided on an inner wall of each supporting arm and is concave towards a direction of an outer wall of the supporting arm; and a width of the sliding slot matches a thickness of a biochip.

A bottom surface of each observation window is made of a transparent material. When the biochip is inserted into the sliding slot, each observation window is aimed at a corresponding sample area on a surface of the biochip. A gap exists between the bottom surface of each observation window and an upper end surface of the biochip, and the gap forms a chip reaction area. The bottom surface of each observation window is made of the transparent material. When the biochip is inserted, the sample area on the biochip is accurately arranged below the corresponding observation window by an operator through the observation window, which enables a solution containing hybridization components to accurately enter the chip reaction area through the sample addition hole and contact a sample on the surface of the biochip, thereby finishing hybridizing and guaranteeing a hybridization success rate.

Preferably, the gap is 0.2-0.5 mm, and the chip reaction area in a range of 0.2-0.5 mm is appropriate.

Preferably, a side of the substrate has an arc concave, for easily taking the biochip.

The first hybridization box comprises a box body, wherein:

An empty cavity is provided in the box body; a side of the box body has an opening for intercommunicating with the empty cavity; and a box cover is movably arranged on the opening for sealing the empty cavity;

A biochip placement box is arranged in the empty cavity; multiple first horizontal slots respectively matching the second hybridization boxes are provided at an upper part of the biochip placement box; the first horizontal slots respectively define the placement cavities; and entrances of the first horizontal slots are provided at the opening;

A second horizontal slot with a width longer than that of the first horizontal slots is provided at a lower part of the biochip placement box; an entrance of the second horizontal slot is provided at the opening; and a humidifying plate is horizontally movably arranged in the second horizontal slot; and heating plates and a heating circuit are arranged in the box body; the heating plates are connected with the heating circuit; and, a USB (Universal Serial Bus) charging plug is arranged outside the box body and connected with the heating circuit.

Both of the box body and the box cover of the first hybridization box are made of a thermal insulation material which has a relatively good heat preservation effect.

The first hybridization box has a simple structure. The opening is provided at one side of the box body, for easily placing and taking the second hybridization boxes and the humidifying plate. The box cover is for sealing the empty cavity; so as to provide a constant environment for hybridization of the biochip. The biochip placement box is arranged in the empty cavity of the box body, wherein: multiple first horizontal slots respectively matching the second hybridization boxes are provided at the upper part of the biochip placement box, and the second hybridization boxes are horizontally movably arranged in the respective first horizontal slots, which has a simple structure and is easy for taking and placing the second hybridization boxes; meanwhile, the first horizontal slots provide a stable placement space for the second hybridization boxes, which is able to effectively avoid shaking of the second hybridization boxes during a hybridization process, thereby effectively guaranteeing the hybridization success rate; the second horizontal slot is provided at the lower part of the biochip placement box; the humidifying plate is horizontally movably arranged in the second horizontal slot, and a constant-humidity hybridization environment is provided for the hybridization of the biochip through the humidifying plate; the heating plates and the heating circuit are arranged in the box body; a constant-temperature hybridization environment is provided for the hybridization of the biochip through the heating plates and the related heating circuit; the USB charging plug is arranged outside the box body, and the heating circuit is charged through the USB charging plug, which has a simple structure and a convenient operation.

Upper surfaces of the entrances of the first horizontal slots and the second horizontal slot have an arc convex part which is convex upward; lower surfaces of the entrances of the first horizontal slots and the second horizontal slot have an arc concave part which is concave downward; and a position of the arc convex part is opposite to a position of the corresponding arc concave part. Structures of the arc convex parts and the arc concave parts are convenient for taking the second hybridization boxes and the humidifying plate.

A lower part of the box cover is connected to a lower part of the side where the opening is provided through a hinge; a fastener is arranged on an inner surface of an upper part of the box cover; a fastener slot, matching the fastener, is provided at a contact part of the box body, wherein the contact part is for contacting the box cover; when closing the box cover, the fastener is inserted into the fastener slot; and a seal ring is arranged at the contact part of the box body. Connection and closing manners of the box cover are easy, a structure thereof is simple, and an operation thereof is convenient. The seal ring arranged at the contact part of the box body, is for guaranteeing a sealing condition of an interior of the box body when closing the box cover, and ensuring sealing of the hybridization environment.

Air gaps exist between the upper part and the lower part of the biochip placement box; and a center area of the humidifying plate is concave downward. Through the air gaps, wet gas generated by the humidifying plate is relatively well provided for the second hybridization boxes, so as to relatively well meet hybridization conditions; and, the concave-downward center area of the humidifying plate is for containing water or wet absorbent paper.

Alternatively, the first hybridization box comprises the box body and the box cover arranged on the box body, wherein: the box body comprises a base plate and an accommodating section arranged on the base plate; a front supporting seat and a back supporting seat, having a same size, are transversely arranged in the accommodating section in parallel; an interval exists between the front supporting seat and the back supporting seat; a back end surface of the front supporting seat, a front end surface of the back supporting seat, and inner walls at two sides of the accommodating section form a center cavity; a humidifying device is arranged in the accommodating section; multiple front isolation blocks are vertically arranged on the front supporting seat with an interval; the front isolation blocks are uniformly distributed on an upper end surface of the front supporting seat; multiple back isolation blocks are vertically arranged on the back supporting seat with an interval; the back isolation blocks are uniformly distributed on an upper end surface of the back supporting seat; one placement cavity is formed among two adjacent front isolation blocks and two opposite adjacent back isolation blocks; the heating plate and the heating circuit are arranged in the base plate; the heating plate is connected with the heating circuit; the USB charging plug is arranged outside the box body and connected with the heating circuit. The above-described first hybridization box has a simple structure. One placement cavity is formed among two adjacent front isolation blocks and two opposite adjacent back isolation blocks, on which the second hybridization box is arranged, and the placement cavity provides a stable placement space for the second hybridization box. The humidifying device is arranged in the accommodating section; and, through the humidifying device, a constant-humidity hybridization environment is provided for the hybridization of the biochip. The heating plate and the heating circuit are arranged in the base plate; and a constant-temperature hybridization environment is provided for the hybridization of the biochip through the heating plate and the heating circuit. The USB charging plug is arranged outside the box body; and through the USB charging plug, the heating circuit is charged, which has a simple structure and a convenient operation.

The humidifying device is a water absorbing sponge. Enough water is absorbed by the water absorbing sponge, and the water absorbing sponge is arranged in the accommodating section, for guaranteeing a relatively constant humidity in the first hybridization box is maintained during heating, so as to meet the hybridization conditions.

Horizontal widths between two adjacent front isolation blocks and between two adjacent back isolation blocks match a width of the second hybridization box, and a distance between the back end surface of the front supporting seat and the front end surface of the back supporting seat is smaller than a length of the second hybridization box, which guarantees the second hybridization box is able to be stably arranged on the placement cavity.

The seal ring is arranged at a contact part of the box body, wherein the contact part is for contacting the box cover. The seal ring is for guaranteeing a sealing condition of the interior of the box body when closing the box cover, so as to guarantee sealing of the hybridization environment.

Compared with prior art, the present invention has following advantages. The second hybridization boxes of the hybridization device have a simple structure. The supporting arms are respectively arranged at the two sides of the bottom surface of the substrate and vertical to the substrate; the sliding slots matching a thickness of the biochip are respectively provided on the inner walls of the supporting arms, and when using, the biochip is inserted into the sliding slots and arranged below the substrate; a stable support is provided for the biochip through the supporting arms, which effectively avoids sliding of the biochip and guarantees the hybridization success rate; the first hybridization box provides a constant hybridization space for the hybridization of the biochip, without any additional auxiliary equipment, which saves a cost; and multiple observation windows, which are concave downward, are arranged on the substrate, which realizes a simultaneous hybridization of multiple biochips.

These and other objectives, features, and advantages of the present invention will become apparent from the following detailed description, the accompanying drawings, and the appended claims.

### BRIEF DESCRIPTION OF THE DRAWINGS

FIG. 1 is a structural sketch view of a second hybridization box according to a first preferred embodiment of the present invention.

FIG. 2 is a structural sketch view of the second hybridization box when a biochip is inserted into the second hybridization box according to the first preferred embodiment of the present invention.

FIG. 3 is a structural sketch view of a first hybridization box according to the first preferred embodiment of the present invention.

FIG. 3a is a sketch view of the first hybridization box when the box cover is closed according to the first preferred embodiment of the present invention.

FIG. 3b is a structural sketch view of heating plates and a related heating circuit according to the first preferred embodiment of the present invention.

FIG. 4 is a sectional view of the first hybridization box according to the first preferred embodiment of the present invention.

FIG. 5 is a structural sketch view of a humidifying plate according to the first preferred embodiment of the present invention.

FIG. 6 is a structural sketch view of a first hybridization box according to a second preferred embodiment of the present invention.

FIG. 7 is a sectional view of a box body of the first hybridization box according to the second preferred embodiment of the present invention.

FIG. 7a is a structural sketch view of a heating plate and a related heating circuit of the first hybridization box according to the second preferred embodiment of the present invention.

FIG. 8 is a structural sketch view of a second hybridization box according to a third preferred embodiment of the present invention.

FIG. 9 is a structural sketch view of the second hybridization box when a biochip is inserted into the second hybridization box according to the third preferred embodiment of the present invention.

### DETAILED DESCRIPTION OF THE PREFERRED EMBODIMENT

The present invention is further described with accompanying drawings and preferred embodiments.

### First Preferred Embodiment

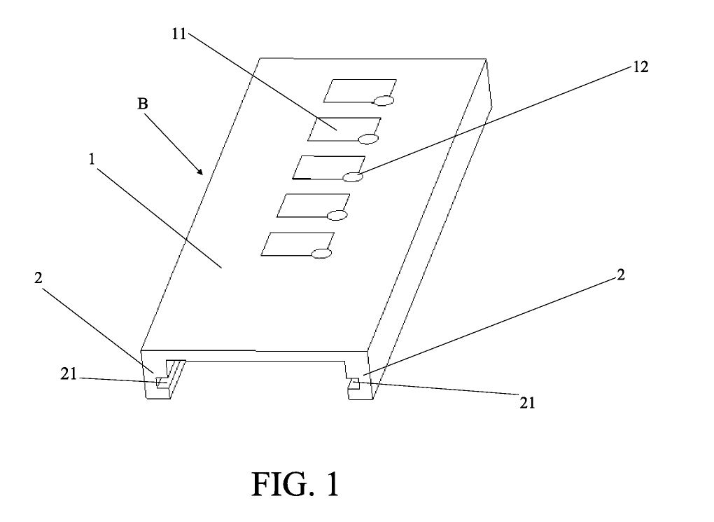

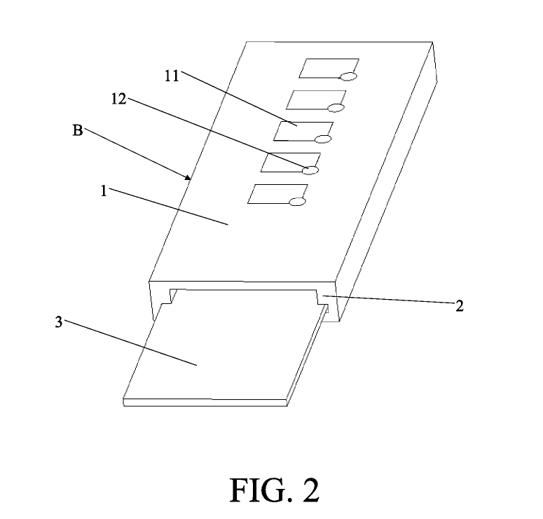

According to the first preferred embodiment of the present invention, a biochip hybridization device comprises a first hybridization box A and second hybridization boxes B, wherein multiple placement cavities for respectively placing the second hybridization boxes B are horizontally provided in the first hybridization box A. As showed in FIG. 1 and FIG. 2, each second hybridization box B comprises a substrate 1 which is horizontally arranged, wherein: multiple observation windows 11, which are concave downward, are arranged on the substrate 1 side by side; a sample addition hole 12, which penetrates through the substrate 1, is provided at an edge of each observation window 11; two supporting arms 2 are respectively arranged at two sides of a bottom surface of the substrate 1 and vertical to the substrate 1; a sliding slot 21 is provided on an inner wall of each supporting arm 2 and is concave towards a direction of an outer wall of the supporting arm 2; and a width of the sliding slot 21 matches a thickness of a biochip 3.

According to the first preferred embodiment, a bottom surface of each observation window 11 is made of a transparent material. When the biochip 3 is inserted into the sliding slot 21, each observation window 11 is aimed at a corresponding sample area on a surface of the biochip 3. A gap exists between the bottom surface of each observation window 11 and an upper end surface of the biochip 3, and the gap forms a chip reaction area. The bottom surface of each observation window 11 is made of the transparent material. When the biochip is inserted, the sample area on the biochip 3 is accurately arranged below the corresponding observation window 11 by an operator through the observation window 11, which enables a solution containing hybridization components to accurately enter the chip reaction area through the sample addition hole 12 and contact a sample on the surface of the biochip 3, thereby finishing hybridizing and guaranteeing a hybridization success rate.

According to the first preferred embodiment, the gap is 0.2 mm.

According to the first preferred embodiment, a bore diameter of each sample addition hole 12 becomes gradually smaller from top to bottom and each sample addition hole 12 is funnel-shaped, so that the sample addition hole 12 matches a tip of a pipette and the tip is easily inserted into the sample addition hole 12, and meanwhile the solution is accurately dropped to the sample area on the surface of the biochip 3.

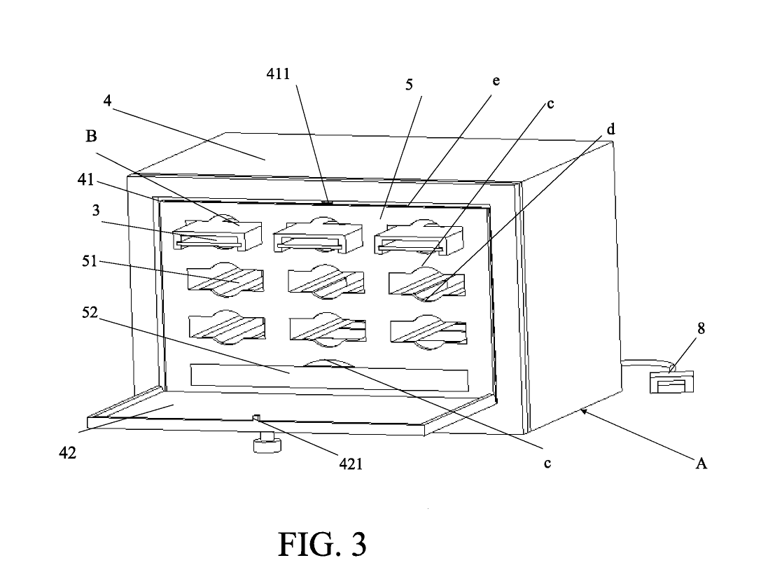

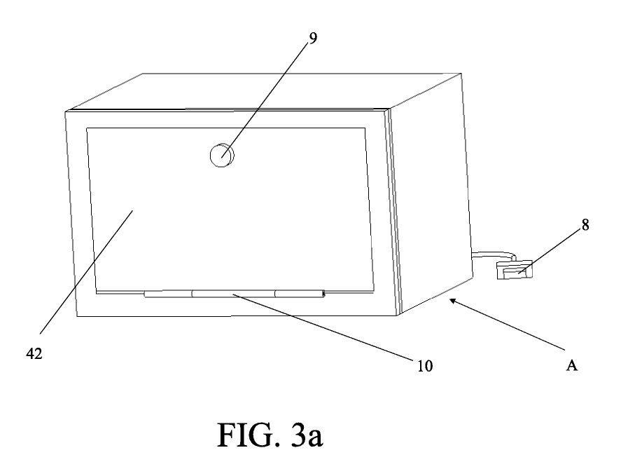

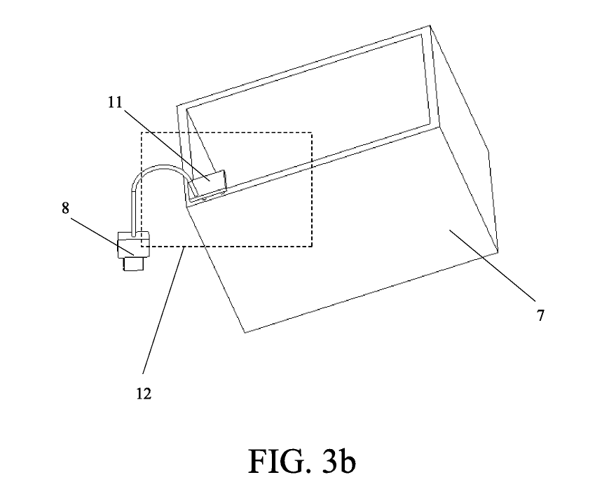

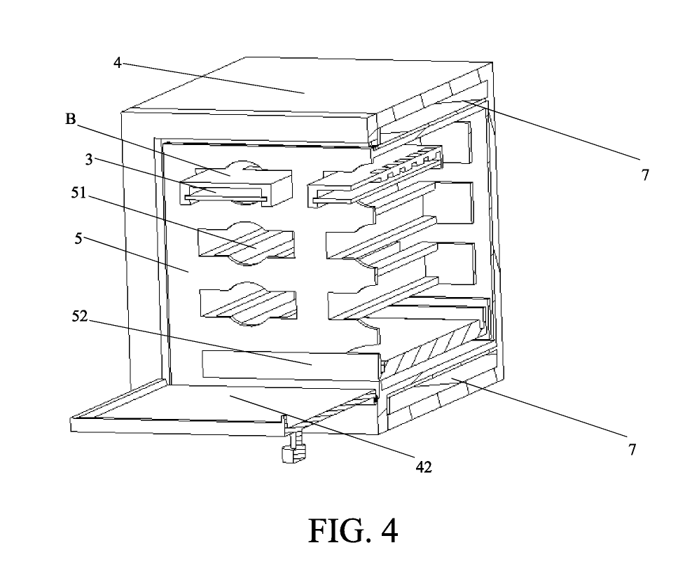

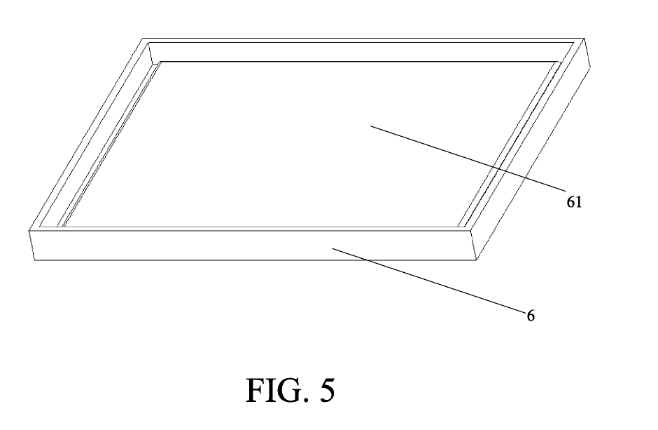

According to the first preferred embodiment, as showed in FIG. 3-FIG. 5, the first hybridization box A comprises a box body 4, wherein: an empty cavity is provided in the box body 4; a side of the box body 4 has an opening 41 for intercommunicating with the empty cavity; and a box cover 42 is movably arranged on the opening 41 for sealing the empty cavity.

According to the first preferred embodiment, a biochip placement box 5 is arranged in the empty cavity; multiple first horizontal slots 51 respectively matching the second hybridization boxes B are provided at an upper part of the biochip placement box 5; the first horizontal slots 51 respectively define the placement cavities for placing the second hybridization boxes B; and entrances of the first horizontal slots 51 are provided at the opening 41.

According to the first preferred embodiment, a second horizontal slot 52 with a width longer than that of the first horizontal slots 51 is provided at a lower part of the biochip placement box 5; an entrance of the second horizontal slot 52 is provided at the opening 41; and a humidifying plate 6 is horizontally movably arranged in the second horizontal slot 52.

According to the first preferred embodiment, heating plates 7 and a heating circuit 12 are arranged in the box body 4; the heating plates 7 are connected with the heating circuit 12; and, a USB (Universal Serial Bus) charging plug 8 is arranged outside the box body 4 and connected with the heating circuit 12.

According to the first preferred embodiment, both of the box body 4 and the box cover 42 of the first hybridization box A are made of a thermal insulation material which has a relatively good heat preservation effect.

According to the first preferred embodiment, a temperature controlling chip 11 is arranged in the heating circuit 12; and through the temperature controlling chip 11, a temperature inside the first hybridization box A is controlled, so as to provide an accurate constant-temperature environment.

According to the first preferred embodiment, the heating plates 7 are arranged around the box body 4, for increasing a heating efficiency, so that the empty cavity has a uniform temperature.

The first hybridization box A has a simple structure. The opening 41 is provided at one side of the box body 4, for easily placing and taking the second hybridization boxes B and the humidifying plate 6. The box cover 42 is for sealing the empty cavity; so as to provide a constant environment for hybridization of the biochip 3. The biochip placement box 5 is arranged in the empty cavity of the box body 4. Multiple first horizontal slots 51 respectively matching the second hybridization boxes B are provided at the upper part of the biochip placement box 5, and the second hybridization boxes B are horizontally movably arranged in the respective first horizontal slots 51, which has a simple structure and is easy for taking and placing the second hybridization boxes B; and meanwhile, the first horizontal slots 51 provide a stable placement space for the second hybridization boxes B, which is able to effectively avoid shaking of the second hybridization boxes B during a hybridization process, thereby effectively guaranteeing the hybridization success rate. The second horizontal slot 52 is provided at the lower part of the biochip placement box 5; the humidifying plate 6 is horizontally movably arranged in the second horizontal slot 52, and a constant-humidity hybridization environment is provided for the hybridization of the biochip 3 through the humidifying plate 6. The heating plates 7 and the heating circuit 12 are arranged in the box body 4; a constant-temperature hybridization environment is provided for the hybridization of the biochip 3 through the heating plates 7 and the related heating circuit 12. The USB charging plug 8 is arranged outside the box body 4 and the heating circuit 12 is charged through the USB charging plug 8, which has a simple structure and a convenient operation.

According to the first preferred embodiment, upper surfaces of the entrances of the first horizontal slots 51 and the second horizontal slot 52 have an arc convex part “c” which is convex upward; lower surfaces of the entrances of the first horizontal slots 51 have an arc concave part “d” which is concave downward; and a position of the arc convex part “c” is opposite to a position of the corresponding arc concave part “d”. Structures of the arc convex parts “c” and the arc concave parts “d” are convenient for taking the second hybridization boxes B and the humidifying plate 6.

According to the first preferred embodiment, a lower part of the box cover 42 is connected to a lower part of the side where the opening 41 is provided through a hinge 10; a fastener 421 is arranged on an inner surface of an upper part of the box cover 42; a fastener slot 411 matching the fastener 421 is provided at a contact part of the box body 4, wherein the contact part is for contacting the box cover 42; when closing the box cover 42, the fastener 421 is inserted into the fastener slot 411; a seal ring “e” is arranged at the contact part of the box body 4; and a handle 9 is arranged for opening and closing the box cover 42. Connection and closing manners of the box cover 42 are easy, a structure thereof is simple, and an operation thereof is convenient. The seal ring “e”, arranged at the contact part of the box body 4, is for guaranteeing a sealing condition of an interior of the box body 4 when closing the box cover 42, and ensuring sealing of the hybridization environment.

According to the first preferred embodiment, air gaps exist between the upper part and the lower part of the biochip placement box 5; a center area 61 of the humidifying plate 6 is concave downward; through the air gaps, wet gas generated by the humidifying plate 6 is relatively well provided for the second hybridization boxes B, so as to relatively well meet hybridization conditions; and the concave-downward center area 61 of the humidifying plate 6 is for containing water or wet absorbent paper.

According to the first preferred embodiment, the upper part of the biochip placement box 5 is divided into multiple rows from top to bottom, and multiple first horizontal slots 51 are horizontally provided at each row of the biochip placement box 5; the second horizontal slot 52 is provided below the bottom first horizontal slot 51; air gaps exist between every two adjacent first horizontal slots 51; through the air gaps, the wet gas generated by the humidifying plate is relatively well provided for the second hybridization boxes B, so as to relatively well meet the hybridization conditions.

### Second Preferred Embodiment

According to the second preferred embodiment of the present invention, a biochip hybridization device comprises a first hybridization box A and second hybridization boxes B, wherein multiple placement cavities for respectively placing the second hybridization boxes B are horizontally provided in the first hybridization box A. As showed in FIG. 1 and FIG. 2, each second hybridization box B comprises a substrate 1 which is horizontally arranged, wherein: multiple observation windows 11, which are concave downward, are arranged on the substrate 1 side by side; a sample addition hole 12, which penetrates through the substrate 1, is provided at an edge of each observation window 11; two supporting arms 2 are respectively arranged at two sides of a bottom surface of the substrate 1 and vertical to the substrate 1; a sliding slot 21 is provided on an inner wall of each supporting arm 2 and is concave towards a direction of an outer wall of the supporting arm 2; and a width of the sliding slot 21 matches a thickness of a biochip 3.

According to the second preferred embodiment, a bottom surface of each observation window 11 is made of a transparent material. When the biochip 3 is inserted into the sliding slot 21, each observation window 11 is aimed at a corresponding sample area on a surface of the biochip 3. A gap exists between the bottom surface of each observation window 11 and an upper end surface of the biochip 3, and the gap forms a chip reaction area. The bottom surface of each observation window 11 is made of the transparent material. When the biochip is inserted, the sample area on the biochip 3 is accurately arranged below the observation window 11 by an operator through the observation window 11, which enables a solution containing hybridization components to accurately enter the chip reaction area through the sample addition hole 12 and contact a sample on the surface of the biochip 3, thereby finishing hybridizing and guaranteeing a hybridization success rate.

According to the second preferred embodiment, the gap is 0.2 mm.

According to the second preferred embodiment, a bore diameter of each sample addition hole 12 becomes gradually smaller from top to bottom and each sample addition hole 12 is funnel-shaped, so that the sample addition hole 12 matches a tip of a pipette and the tip is easily inserted into the sample addition hole 12, and meanwhile the solution is accurately dropped to the sample area on the surface of the biochip 3.

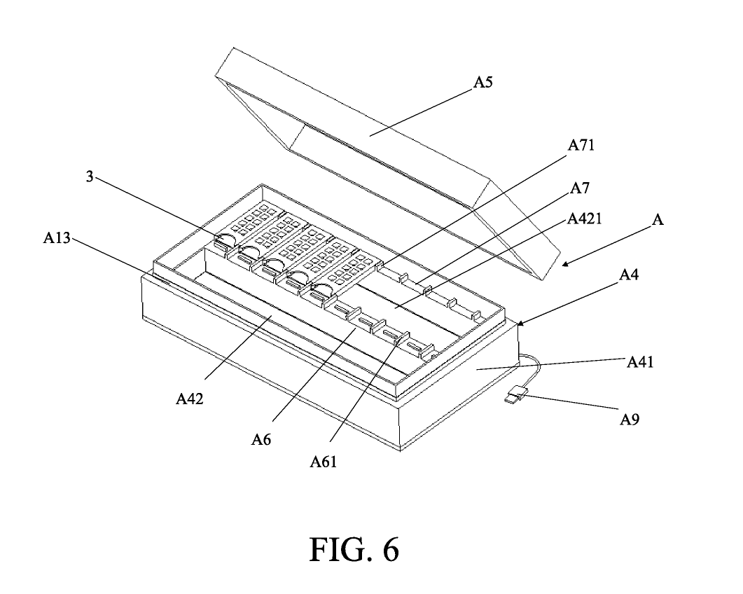

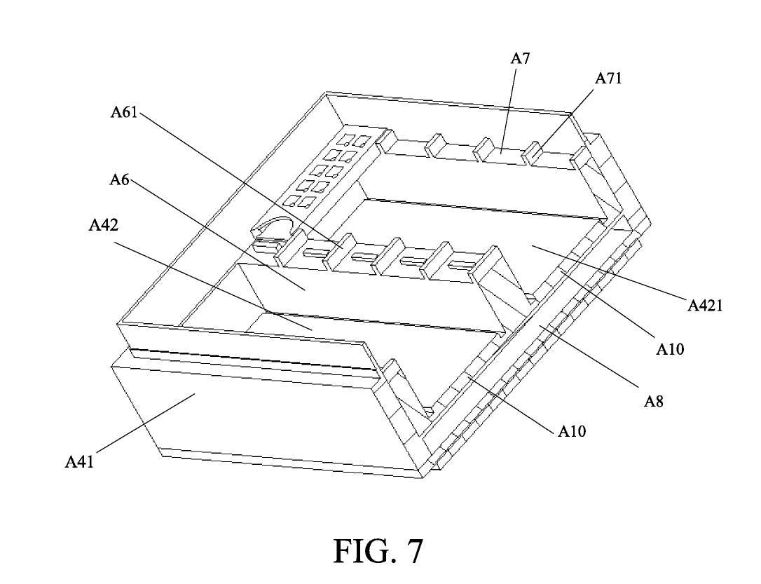

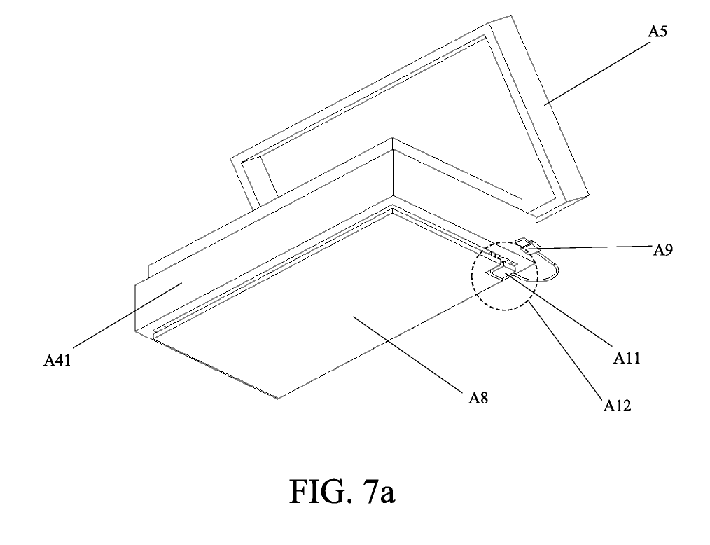

According to the second preferred embodiment, as showed in FIG. 6-FIG. 7a, the first hybridization box A comprises a box body A4 and a box cover A5 arranged on the box body A4, wherein: the box body A4 comprises a base plate A41 and an accommodating section A42 arranged on the base plate A41; a front supporting seat A6 and a back supporting seat A7, having a same size, are transversely arranged in the accommodating section A42 in parallel; an interval exists between the front supporting seat A6 and the back supporting seat A7; a back end surface of the front supporting seat A6, a front end surface of the back supporting seat A7, and inner walls at two sides of the accommodating section A42 form a center cavity A421; a humidifying device A10 is arranged in the accommodating section A42; multiple front isolation blocks A61 are vertically arranged on the front supporting seat A6 with an interval; the front isolation blocks A61 are uniformly distributed on an upper end surface of the front supporting seat A6; multiple back isolation blocks A71 are vertically arranged on the back supporting seat A7 with an interval; the back isolation blocks A71 are uniformly distributed on an upper end surface of the back supporting seat A7; one placement cavity for placing the second hybridization box B is formed among two adjacent front isolation blocks A61 and two opposite adjacent back isolation blocks A71; a heating plate A8 and a heating circuit A12 are arranged inside the base plate A41; the heating plate A8 is connected with the heating circuit A12; and, a USB (Universal Serial Bus) charging plug A9 is arranged outside the box body A4 and connected with the heating circuit A12. The above-described first hybridization box A has a simple structure. One placement cavity is formed among two adjacent front isolation blocks A61 and two opposite adjacent back isolation blocks A71, on which the second hybridization box B is arranged, and the placement cavity provides a stable placement space for the second hybridization box B. The humidifying device A10 is arranged in the accommodating section A42; and, through the humidifying device A10, a constant-humidity hybridization environment is provided for the hybridization of the biochip 3. The heating plate A8 and the heating circuit A12 are arranged inside the base plate A41, and a constant-temperature hybridization environment is provided for the hybridization of the biochip 3 through the heating plate A8 and the heating circuit A12. The USB charging plug A9 is arranged outside the box body A4; and through the USB charging plug A9, the heating circuit A12 is charged, which has a simple structure and a convenient operation.

According to the second preferred embodiment, the humidifying device A10 is a water absorbing sponge. Enough water is absorbed by the water absorbing sponge, and the water absorbing sponge is arranged in the accommodating section A42, for guaranteeing a relatively constant humidity in the first hybridization box A is maintained during heating, so as to meet hybridization conditions.

According to the second preferred embodiment, horizontal widths between two adjacent front isolation blocks A61 and between two adjacent back isolation blocks A71 match a width of the second hybridization box B, and a distance between the back end surface of the front supporting seat A6 and the front end surface of the back supporting seat A7 is smaller than a length of the second hybridization box B, which guarantees the second hybridization box B is able to be stably arranged on the placement cavity.

According to the second preferred embodiment, a seal ring A13 is arranged at a contact part of the box body A4, wherein the contact part is for contacting the box cover A5. The seal ring A13 is for guaranteeing a sealing condition of an interior of the box body A4 when closing the box cover A5, so as to guarantee sealing of the hybridization environment.

According to the second preferred embodiment, a temperature controlling chip A11 is arranged in the heating circuit A12, and a temperature inside the first hybridization box A is controlled through the temperature controlling chip A11, so as to provide an accurate constant-temperature environment.

### Third Preferred Embodiment

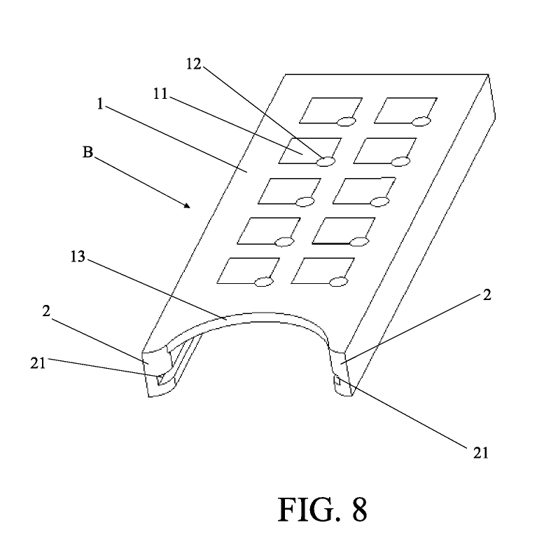

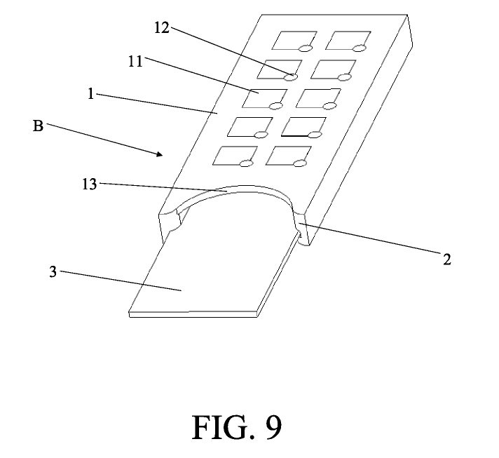

The third preferred embodiment is different from the first preferred embodiment and the second preferred embodiment in that: one side of the substrate 1 has an arc concave 13, for easily taking the biochip 3, as showed in FIG. 8 and FIG. 9.

### Fourth Preferred Embodiment

The fourth preferred embodiment is different from the first preferred embodiment and the second preferred embodiment in that the gap is 0.3 mm.

### Fifth Preferred Embodiment

The fifth preferred embodiment is different from the first preferred embodiment and the second preferred embodiment in that the gap is 0.4 mm.

### Sixth Preferred Embodiment

The sixth preferred embodiment is different from the first preferred embodiment and the second preferred embodiment in that the gap is 0.5 mm.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A biochip hybridization device, comprising a first hybridization box and second hybridization boxes, wherein: multiple placement cavities for respectively placing the second hybridization boxes are horizontally provided in the first hybridization box; each second hybridization box comprises a substrate which is horizontally arranged; multiple observation windows, which are concave downward, are arranged on the substrate side by side; a sample addition hole, which penetrates through the substrate, is provided at an edge of each observation window; two supporting arms are respectively arranged at two sides of a bottom surface of the substrate and vertical to the substrate; a sliding slot is provided on an inner wall of each supporting arm and is concave towards a direction of an outer wall of the supporting arm; and a width of the sliding slot matches a thickness of a biochip.

2. The biochip hybridization device, as recited in claim 1, wherein: a bottom surface of each observation window is made of a transparent material; when the biochip is inserted into the sliding slot, each observation window is aimed at a corresponding sample area on a surface of the biochip; a gap exists between the bottom surface of each observation window and an upper end surface of the biochip, and the gap forms a chip reaction area.

3. The biochip hybridization device, as recited in claim 1, wherein one side of the substrate has an arc concave.

4. The biochip hybridization device, as recited in claim 1, wherein: the first hybridization box comprises a box body, wherein:
An empty cavity is provided in the box body; a side of the box body has an opening for intercommunicating with the empty cavity; and a box cover is movably arranged on the opening for sealing the empty cavity;
A biochip placement box is arranged in the empty cavity; multiple first horizontal slots respectively matching the second hybridization boxes are provided at an upper part of the biochip placement box; the first horizontal slots respectively define the placement cavities; and entrances of the first horizontal slots are provided at the opening;
A second horizontal slot with a width longer than that of the first horizontal slots is provided at a lower part of the biochip placement box; an entrance of the second horizontal slot is provided at the opening; and a humidifying plate is horizontally movably arranged in the second horizontal slot; and heating plates and a heating circuit are arranged in the box body; the heating plates are connected with the heating circuit; and, a USB (Universal Serial Bus) charging plug is arranged outside the box body and connected with the heating circuit.

5. The biochip hybridization device, as recited in claim 4, wherein: upper surfaces of the entrances of the first horizontal slots and the second horizontal slot have an arc convex part which is convex upward; lower surfaces of the entrances of the first horizontal slots and the second horizontal slot have an arc concave part which is concave downward; and a position of the arc convex part is opposite to a position of the corresponding arc concave part.

6. The biochip hybridization device, as recited in claim 4, wherein: a lower part of the box cover is connected to a lower part of the side where the opening is provided through a hinge; a fastener is arranged on an inner surface of an upper part of the box cover; a fastener slot, matching the fastener, is provided at a contact part of the box body, wherein the contact part is for contacting the box cover; when closing the box cover, the fastener is inserted into the fastener slot; and a seal ring is arranged at the contact part of the box body.

7. The biochip hybridization device, as recited in claim 4, wherein air gaps exist between the upper part and the lower part of the biochip placement box; and a center area of the humidifying plate is concave downward.

8. The biochip hybridization device, as recited in claim 1, wherein: the first hybridization box comprises a box body and a box cover arranged on the box body; the box body comprises a base plate and an accommodating section arranged on the base plate; a front supporting seat and a back supporting seat, having a same size, are transversely arranged in the accommodating section in parallel; an interval exists between the front supporting seat and the back supporting seat; a back end surface of the front supporting seat, a front end surface of the back supporting seat, and inner walls at two sides of the accommodating section form a center cavity; a humidifying device is arranged in the accommodating section; multiple front isolation blocks are vertically arranged on the front supporting seat with an interval; the front isolation blocks are uniformly distributed on an upper end surface of the front supporting seat; multiple back isolation blocks are vertically arranged on the back supporting seat with an interval; the back isolation blocks are uniformly distributed on an upper end surface of the back supporting seat; one placement cavity is formed among two adjacent front isolation blocks and two opposite adjacent back isolation blocks; a heating plate and a heating circuit are arranged in the base plate; the heating plate is connected with the heating circuit; a USB (Universal Serial Bus) charging plug is arranged outside the box body and connected with the heating circuit.

9. The biochip hybridization device, as recited in claim 8, wherein: horizontal widths between two adjacent front isolation blocks and between two adjacent back isolation blocks match a width of the second hybridization box, and a distance between the back end surface of the front supporting seat and the front end surface of the back supporting seat is smaller than a length of the second hybridization box.

10. The biochip hybridization device, as recited in claim 8, wherein: a seal ring is arranged at a contact part of the box body, wherein the contact part is for contacting the box cover.
